(19) United States
(12) Patent Application Publication
(10) Pub. No.: US 2011/0282228 A1
(43) Pub. Date: Nov. 17, 2011

(54) Methods and Devices for Determining Pulmonary Measurements

(75) Inventors: Robert Shiner, Tel Aviv (IL); Avi Lazar, Ra'anana (IL); Ori Adam, Rechovot (IL)

(73) Assignee: PULMONE ADVANCED MEDICAL DEVICES, LTD., Ra'anana (IL)

(21) Appl. No.: 13/146,295

(22) PCT Filed: Jan. 28, 2010
(86) PCT No.: PCT/IL2010/000070

§ 371 Date: Jul. 26, 2011

Related U.S. Application Data
(60) Provisional application No. 61/147,787, filed on Jan. 28, 2009.

(51) Int. Cl. A61B 5/091; A61B 5/11; A61B 5/08

(52) U.S. Cl. 600/534; 600/529; 600/538

(57) ABSTRACT

A method for determining at least one instantaneous pulmonary measurement. The method comprises providing a plurality of instantaneous mechanical pulmonary properties of a user in at least one identified respiratory instant during at least one respiration cycle, correlating between the plurality of instantaneous mechanical pulmonary properties, and determining at least one instantaneous pulmonary measurement in at least one identified respiratory instant according to the correlation during the at least one respiration cycle.

### RELATED APPLICATIONS

The content of International Patent Application No. PCT/IL2008/001031 filed on Jul. 27, 2008, is incorporated herein by reference as if fully set forth herein.

This application claims priority from U.S. Patent No. 61/147,787, filed on Jan. 28, 2009.

### FIELD AND BACKGROUND OF THE INVENTION

The present invention, in some embodiments thereof, relates to methods and devices for pulmonary diagnosis and, more particularly, but not exclusively, to methods and devices for determining pulmonary measurements.

The driving force for the respiratory airflow in the airways is the pressure gradient between the lungs and the atmosphere, which is produced by virtue of the pressure change in the alveoli arising from the rhythmic respiratory motion of the chest. The rate of this airflow is directly proportional to the pressure gradient and to the cross section of the respiratory wave, while being inversely proportional to the length of the airway and to the viscosity of the gas. The length and cross section of the respiratory pathways, as well as the texture and properties of the surface interface with the moving air determine the airway resistance.

These values are used diagnostically in clinical and experimental physiology; it is particularly useful clinically to measure the alveolar pressure under certain specific conditions, such as when there is additional resistance to respiration, during gravitational overloading, respiration under elevated or reduced pressures, inspiration of gas mixtures differing in density from air, and so on.

One of the most common methods for measuring alveolar pressure is the airway interruption method, see Sawashima, M., Honda, K., Niimi, S. & Hirose, H. [1986], some clinical data on aerodynamic examination using the airway interruption method. Ann. Bull. RILP, 20, 217-224, which is incorporated herein by reference. This method is commonly utilized for estimating the resistance of the lower respiratory tract in pulmonary diseases. In this method, respiratory airflow is momentarily interrupted by a shutter attached to the mouth of the user. The air pressure at the occluded airways is measured at the moment when it reaches complete equilibrium with the alveolar pressure.

For example, U.S. Pat. No. 5,233,998, issued on Aug. 10, 1993 describes an apparatus for measuring airway resistance, which apparatus comprises a housing, a bore in the housing, a flow measuring device which is positioned in the bore and which is for measuring a flow of air through the bore caused by a person exhaling or inhaling, an interrupting valve device which is for interrupting the flow of air through the bore, a drive device for moving the interrupting valve device between a bore closing position and a bore opening position, and a pressure sensor device for sensing variations in pressure in the bore caused by the interrupting valve device closing and opening the bore and thus interrupting the flow of air through the bore, and the apparatus being such that the bore is of circular cross section, and the interrupting valve device is a non-circular elliptical plate member which is mounted in the bore for pivotal movement by the drive device between the bore closing position and the bore opening position and which extends at an acute angle to the bore in the bore closing position.

Another example is described in International patent Application No. PCT/IL2008/001031 filed on Jul. 27, 2008 disclosing a system and method for determining Functional Residual Capacity (FRC), Total Lung Capacity (TLC) and Residual Volume (RV) includes a hand-held unit with a shutter assembly designed to minimize measured air displacement due to shuttering. Measurements of airflow and pressure are used to derive the lung parameters.

Another value important when assessing lung function is pulmonary volume. Nowadays, the Body Plethysmograph is the most common method used for measuring pulmonary volumes. In practice, the subject sits in a large airtight box; where pressure and volume changes are measured according to the principle that at constant temperature and at adiabatic conditions, the volume of a gas varies inversely as the absolute pressure applied to the gas during respiratory efforts.

The calculation of the volume and the pressure facilitates a plot of a Respiratory System Compliance curve, a pressure-volume curve for the lung or relaxed chest wall; plotting volume as a function of pressure inside minus pressure outside. The slope of this curve is the compliance. Respiratory System Compliance, denoted herein as C_{RS}, is usually calculated as follows:

$${C}_{RS} = \frac{{V}_{a} - {V}_{b}}{{P}_{a} - {P}_{b}}$$

where V and P denote, respectively, the lung volume and pressure under static conditions and the indices a and b denote different static lung states where Vₐ represent a larger volumetric size than V_{b}. Respiratory system compliance may be understood as a measure of the tendency of the respiratory system to resist recoil toward base dimensions upon removal of a distending inhaled air or compressing muscle force. Commonly, respiratory system compliance is measured under static conditions, but it may be estimated during continuous breathing.

Respiratory system compliance may assist in management and diagnosis of patients sharing similar symptoms, such as hyperinflation, airway obstruction and expiratory flow limitation but with different respiratory disorders. For example, respiratory system compliance is used to differentiate between emphysema which results in high respiratory system compliance during relaxed breathing, and fibrotic lung conditions which are characterized by low respiratory system compliance, even during relaxed breathing. In the aforementioned equation, C_{RS} is calculated according to the value of Vₐ and V_{b} and not only on the difference between them. Respiratory system compliance varies according to the measured respiratory instant during the respiration cycle. For clarity, as used herein a respiration cycle means one or more circulatory exhalations and inhalations. For example, a respiration cycle can be considered to start at substantially full tidal exhalation and end at substantially full tidal inhalation. Optionally, the respiration cycle includes a respiration hold introduced before, during, and/or after the exhalation and/or inhalation. The respiration cycle may be a natural respiration cycle as well as an artificial respiration cycle. The respiration cycle may be a tidal breathing respiration cycle, a breathing respiration cycle on exertion, and/or guided respiration cycle. The respiration cycle may be full inspiration to TLC level and full expiration to RV level by either a forced or a slow maneuver.

The pressure-volume relation of the respiratory system is non-linear. In general, with respect to volume, the respiratory system compliance increases between the RV level and FRC level, is approximately constant at the tidal breathing range and decreases above the tidal breathing range. Consequently, C_{RS} is sensitive to the difference between levels a and b in the above equation, in particular outside the tidal breathing range. For example, for a given V_{b} at the FRC level, if Vₐ is close to the Functional Residual Capacity (FRC) level, C_{RS} is relatively large and when Vₐ is equal or substantially equal to the Total Lung Capacity (TLC), the measured C_{RS} is relatively small. Therefore, between the FRC and TLC levels, C_{RS} is affected by the inverse of the actual lung volume at which it is measured which affects the rate of change of the volume with respect to lung pressure at that level.

### SUMMARY OF THE INVENTION

According to some embodiments of the present invention there is provided a method for determining at least one instantaneous pulmonary measurement. The method comprises a) providing a plurality of instantaneous mechanical pulmonary properties of a user in at least one identified respiratory instant during at least one respiration cycle, b) correlating between the plurality of instantaneous mechanical pulmonary properties, and c) determining at least one instantaneous pulmonary measurement in the at least one identified respiratory instant according to the correlation during the at least one respiration cycle.

Optionally, a first of the instantaneous mechanical pulmonary properties is an instantaneous alveolar pressure.

Optionally, a first of the instantaneous mechanical pulmonary properties is an instantaneous pulmonary volume.

Optionally, a)-c) are performed without cooperation of the user.

Optionally, the at least one respiration cycle are tidal breathing cycles.

Optionally, the method further comprises outputting the at least one instantaneous pulmonary measurement during the at least one respiration cycle.

Optionally, the at least one instantaneous pulmonary measurement comprises an estimation of respiratory system compliance in the at least one identified respiratory instant.

More optionally, the at least one respiration cycle comprising an exhalation stage and an inhalation stage, the determining comprises determining at an expiratory respiratory system compliance estimation based on the exhalation stage and an inspiratory respiratory system compliance estimation based on the inhalation stage and calculating a ratio between the expiratory and inspiratory respiratory system compliance estimations.

More optionally, the at least one respiration cycle is a tidal breathing respiration cycle.

More optionally, the user is in a static respiratory state during the at least one respiration cycle.

Optionally, the at least one instantaneous pulmonary measurement comprises an estimation of a pulmonary airway resistance in the at least one identified respiratory instant.

Optionally, the user performs the at least one respiration cycle according to a pulmonary function test (PFT).

More optionally, at least one of the plurality of instantaneous mechanical pulmonary properties is a continuous testing data of the PFT with respect to the at least one identified respiratory instant.

More optionally, the PFT is a spirometry test, wherein a first of the instantaneous mechanical pulmonary properties comprises at least one of a volume-time curve and a flow-volume loop.

Optionally, at least one of the plurality of instantaneous mechanical pulmonary properties is a negative expiratory pressure (NEP) test result.

More optionally, the determining comprises adjusting a second of the plurality of instantaneous pulmonary properties according to the NEP test result.

Optionally, the method further comprises monitoring a continuous respiration of the user by repeating the a)-c) during a plurality of respiratory instants during the at least one respiration cycle.

More optionally, the monitoring comprises generating an alert according to the at least one instantaneous pulmonary measurement.

More optionally, a) comprises measuring a flow rate of a user during at least one respiration cycle and identifying a plurality of reference instants therein according to at least one known respiratory airflow pattern and using the flow rate during the plurality of reference instants for estimating the instantaneous pulmonary volume.

More optionally, a) comprises allowing a user to breathe during the at least one respiration cycle via a conduit and measuring a flow rate in the conduit for estimating the instantaneous pulmonary volume.

More optionally, the user performs the at least one respiration cycle according to an endurance test, and a second of the plurality of instantaneous mechanical pulmonary properties is an endurance test value. Moreover a) may comprise adjusting a resistance element in the conduit for applying various resistance levels on an airflow induced by the breathing.

Optionally, the method further comprises dispensing a chemical according to the at least one instantaneous pulmonary measurement.

Optionally, the method further comprises allowing a user to inhale the chemical, such as oxygen, during the at least one respiration cycle.

Optionally, the method further comprises detecting at least one pulmonary manifestation of at least one cardiac disorder according to the at least one instantaneous pulmonary property.

Optionally, the method further comprises assessing a pulmonary manifestation of at least one of a cardiac disorder, respiratory muscle strength disorder, and a pulmonary disorder according to the instantaneous pulmonary measurement.

According to some embodiments of the present invention there is provided an apparatus for determining at least one instantaneous pulmonary measurement. The apparatus comprises a housing defining a chamber having at least one of a mouthpiece, an endotracheal tube and a mask configured for allowing a user to exhale and inhale in at least one respiration cycle via the chamber, at least one sensor positioned in the chamber and configured for measuring a plurality of mechanical pulmonary properties according to the exhaling and inhaling, a control module configured to generate at least one instantaneous pulmonary measurement by correlating between the plurality of mechanical pulmonary properties, and an output unit configured to output the at least one instantaneous pulmonary measurement during the at least one respiration cycle.

Optionally, the apparatus further comprises a shutter configured for occluding and reopening at least a portion of the chamber and a pump for propelling air from the portion or into the portion, the at least one sensor being configured for measuring a flow rate during the reopening. The propelling may be performed before the reopening and the control module may be configured to detect an expiratory flow limitation (EFL) of the user according to the flow rate.

Optionally, the chamber may have a configurable element for adjusting an airflow resistance in the chamber during the exhaling and inhaling.

Optionally, the pump is located between the mouth of the user and the shutter.

Optionally, the shutter is located between the mouth of the user and the pump.

Optionally, the chamber is connected to an external container of varying volumetric capacity.

Optionally, the pump injects or extracts air from the portion at a rate that is significantly faster than the airflow rate of respiration at the instant of measurement.

Optionally, the apparatus further comprises a communication unit configured to forward the at least one instantaneous pulmonary measurement to a client terminal.

Optionally, the apparatus further comprises a presentation unit configured to continuously present the at least one instantaneous pulmonary measurement.

Optionally, the apparatus further comprises a presentation unit configured to present instructions for respiration during the at least one respiration cycle.

More optionally, the instructions are selected according to at least one of medical information and a medical history pertaining to the user.

Optionally, the apparatus further comprises a memory unit configured to record the at least one instantaneous pulmonary measurement.

Optionally, the apparatus further comprises a dispenser configured to dispense a chemical to the chamber according to the at least one instantaneous pulmonary measurement.

Optionally, the apparatus further comprises at least one biological measurement unit for measuring at least one biological parameter of the user during the at least one respiration cycle, and the control module may be configured to adjust the at least one instantaneous pulmonary measurement according to the at least one biological parameter.

More optionally, the at least one biological measurement unit may comprise a member of a group consisting of: a pulsometer, a pulse oximeter, a sphygmomanometer, an image sensor, an audio sensor, a thermometer, a perspiration sensor, and a brain activity sensor.

According to some embodiments of the present invention there is provided an apparatus for determining at least one instantaneous pulmonary measurement. The apparatus comprises a housing defining a chamber having at least one of a mouthpiece an endotrachel tube and a mask configured for allowing a user to exhale and inhale in at least one respiration cycle via the chamber, at least one sensor positioned in the chamber and configured for measuring a plurality of mechanical pulmonary properties according to the exhaling and inhaling, a control module configured for generating an instantaneous pulmonary volume and an instantaneous alveolar pressure in a plurality of respiratory instants of the at least one respiration cycle and calculating a respiratory system compliance estimation in each the respiratory instant accordingly, and an output unit configured for outputting the respiratory system compliance during the at least one respiration cycle.

Optionally, the housing is a handheld housing.

Optionally, the control module may be configured to calculate an airway resistance in each the respiratory instant according to the instantaneous pulmonary volume and the instantaneous alveolar pressure, and an output unit may be configured to output the airway resistance.

Optionally, the apparatus further comprises a communication unit configured to establish a communication session with a terminal and to forward at least one of the respiratory system compliance, the pulmonary volume, the instantaneous alveolar pressure and derivatives thereof, to a client terminal thereto.

More optionally, the communication session is established via a client terminal from a group consisting of: a cellular phone, a personal computer, a Smartphone and a palm.

Optionally, the apparatus is adapted for veterinary use.

Optionally, the apparatus can be mobilized, and operate while being mobilized, such as for example accompany a mobilized patient recovering from surgery or a mobilized patient restricted to a wheelchair.

Optionally, the apparatus is adapted for bedside use.

Optionally, the apparatus is adapted for measuring patients in a supine or seated position.

Optionally, the apparatus is adapted for measuring patients while being in a state of exercise, such as in cardiac challenge tests.

Optionally, the apparatus is adapted for invasively and non-invasively mechanically ventilated patients. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a method for determining one or more instantaneous pulmonary measurements, according to some embodiments of the present invention;

FIG. 2 is a graph depicting pulmonary volume change pertaining to a user that is breathing according to such a breathing pattern;

FIG. 3 is a schematic illustration of an exemplary breathing apparatus for determining one or more instantaneous pulmonary measurements, according to some embodiments of the present invention;

FIG. 4 is a flowchart of a method for allowing a user to operate the breathing device to perform selected pulmonary tests, according to some embodiments of the present invention;

FIGS. 5A-5C are graphs depicting exemplary instantaneous pulmonary volumes plotted against expiratory tidal respiratory system compliance index and a respective estimated alveolar pressure in a respective respiratory instant;

FIG. 6A is a table that lists expiratory and inspiratory averaged tidal respiratory system compliance index of users of varying ages and medical conditions;

FIG. 6B is a graph of an exemplary airflow pressure curve shown as a solid line and an averaged pressure level shown as a dashed line during an airflow interruption, according to some embodiments of the present invention.

FIG. 7 is a schematic illustration of the breathing device for performing controlled injection or extraction of air, also enabling a negative expiratory pressure (NEP) test while measuring instantaneous alveolar pressure and/or volume, according to some embodiments of the present invention; and

FIG. 8 depicts a dependence of airway resistance on the instantaneous airflow calculated using the breathing device.

### DESCRIPTION OF EMBODIMENTS OF THE INVENTION

The present invention, in some embodiments thereof, relates to methods and devices for pulmonary diagnosis and, more particularly, but not exclusively, to methods and devices for determining pulmonary measurements.

According to some embodiments of the present invention there is provided a method and an apparatus for determining one or more instantaneous pulmonary measurements, such as respiratory system compliance, at one or more respiratory instances during one or more respiration cycles, such as tidal respiration cycles. The method is based on instantaneous mechanical pulmonary properties, such as instantaneous alveolar pressure and/or pulmonary volume which are calculated in one or more respiratory instants during one or more respiration cycles. The instantaneous mechanical pulmonary properties are correlated. The correlating of the instantaneous pulmonary volume may allow for the determination of one or more instantaneous pulmonary measurements, such as instantaneous respiratory system compliance, instantaneous airway resistance and Negative Expiratory Pressure (NEP) testing, according to the correlated instantaneous pulmonary volume.

Optionally, the one or more respiration cycles are respiration maneuvers which are performed during a pulmonary related or pulmonary function test (PFT), such as spirometry and cardio and/or respiratory endurance tests, respiratory muscle strength and endurance tests, and cardio-pulmonary exercise tests. In such an embodiment, the instantaneous pulmonary measurements may be PFT performance data that is correlated with a respective instantaneous pulmonary volume and/or instantaneous alveolar pressure.

Optionally, the one or more respiration cycles are tidal respiration cycles.

According to some embodiments of the present invention there is provided a breathing apparatus for determining one or more instantaneous pulmonary measurements. The apparatus, which is optionally a handheld device, comprises a housing that defines a chamber having a mouthpiece, an endotracheal tube or a mask that allows a user to exhale and/or inhale in one or more respiration cycles via the chamber. The device comprises one or more sensors, which are optionally positioned in the chamber. The sensors are configured for measuring mechanical pulmonary properties, such as pressure and airflow rate which are induced in the chamber when the user exhales and/or inhales through it. The apparatus further comprises a control module for determining one or more instantaneous pulmonary measurements in respiratory instants during the aforementioned respiration cycles, for example according to the airway pressure and airflow rate. The apparatus further comprises an output unit for outputting the instantaneous pulmonary property during the respective respiration cycles.

Optionally, the apparatus is designed to perform a negative expiratory pressure (NEP) test during the aforementioned respiration cycles. In such a manner, the device may detect expiratory flow limitations (EFLs) and optionally adjust the instantaneous pulmonary property accordingly.

Optionally, the apparatus is designed to monitor or perform a measurement of low and/or non cooperative patients, such as intensive care patients, patients under general anesthesia, for example during a surgery and children particularly of pre-school age.

Optionally, the apparatus is designed to monitor or perform a measurement of invasively or non-invasively mechanically ventilated patients.

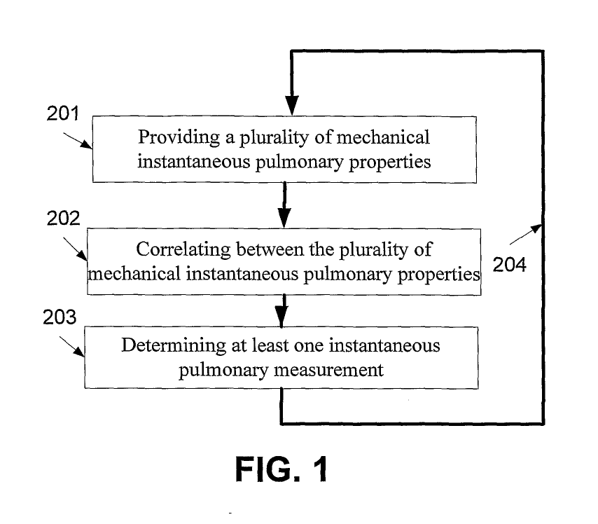

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. Reference is now made to FIG. 1, which is a method for determining one or more instantaneous pulmonary measurements, such as a respiratory system compliance estimation, in one or more respiratory stages or respiratory instants of a respiration cycle of a user, according to some embodiments of the present invention. As used herein, a user means a healthy user, a subject, or a patient to whom the instantaneous pulmonary measurements are related to her lungs, medical condition and/or respiration. The user may be a person or an animal operating a breathing device, for example as described below, and/or a healthy user.

A component of the respiratory system compliance, in particular during relaxed breathing, is the alveolar tissue compliance. The relative contribution of alveolar tissue to the total respiratory system compliance is high when muscle contribution is neutralized, for example, in mechanically ventilated patients, but is also significant during relaxed, normal breathing of normal subjects and patients suffering from pulmonary disorders.

As shown at 201, a number of instantaneous mechanical pulmonary properties at a selected respiratory instant are provided. As used herein a respiratory instant or respiratory stage might refer to a timed instant and/or stage and/or otherwise identified instant and/or stage during a respiration of a user, for example during exhalation, inhalation, between exhalation and inhalation, and/or during a breathing interruption or interlude. As used herein, an instantaneous mechanical pulmonary property might refer to an instantaneous pulmonary volume, an instantaneous alveolar pressure, an instantaneous PFT outcome or estimation, and/or an EFL estimation and the like.

Optionally, one of the instantaneous mechanical pulmonary properties is an instantaneous pulmonary volume, or a density related pulmonary volume change. Optionally, the instantaneous pulmonary volume or volume change may be determined based on airway pressure and flow rate that are measured during and/or after a respiratory modulation, such as a respiration interruption, for example an airway occlusion, for example as described in International Patent Application No. PCT/IL00081/01031 filed on Jul. 27, 2008, which is incorporated herein by reference as if fully set forth herein.

The instantaneous pulmonary volume may be estimated according to one or more reference pulmonary volumes in known respiratory instants, such as RV, FRC, TLC and/or any other respiratory instant having an indicative pattern that may be identified by measuring changes of a respiratory parameter, such as the flow rate in the airway of the user.

Optionally, in order to identify the reference pulmonary volume, the user is instructed to reach one or more known reference respiratory stages along a respiration cycle, identifying a respiratory instant according to its pattern, and the reference pulmonary volume may be measured at this known respiratory instant. This estimation may be performed in real time, during the respiration of the user and/or based on a recording of respiratory parameters, such as airway flow rate. For example, known respiratory reference stages may be RV, thoracic gas volume (TGV) or TLC. These respiratory reference stages may be identified by analyzing the flow rate in the airway of the user and detecting indicative patterns, such as repetitive flow rate waves, a steep flow rate increase and incline, and/or a steep increase.

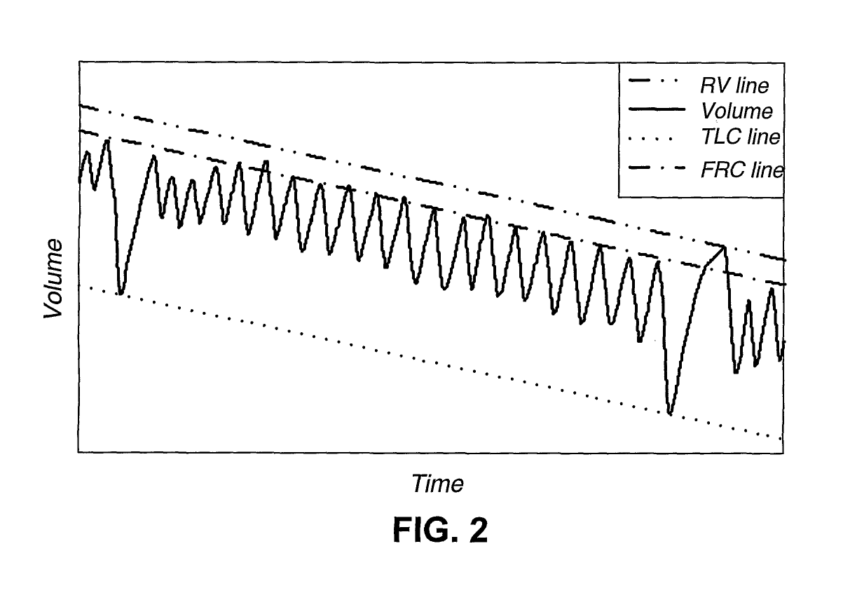

Optionally, in order to validate the volume estimation, the user is instructed in a breathing pattern that incurs repeating a known respiratory reference stage, such as TLC and RV a number of times. In such a manner, drifts and/or trends in the reading of the air flow according to which the instantaneous reference pulmonary volume is calculated may be reduced or removed. For example, FIG. 2 depicts pulmonary volume change pertaining to a user that is breathing according to such a breathing pattern. The breathing pattern causes inflating and/or deflating of the lungs so that their volume is changed with respect to the various repetitive respiratory stages. In FIG. 2, the breathing pattern instructs the user to reach the TLC stage at the beginning of the measurement, then breathe tidally and end with a vital capacity (VC) breathing maneuver. In such a breathing pattern, the user reaches the FRC several times and the TLC level at the beginning and the end of the measurement. This breathing pattern allows for determining the airflow that is contributed by the drift and reducing it from the respective instantaneous pulmonary volume. It is to be noted that drifts may result from a sensor bias or leakage. However, under properly functioning condition, the major contribution to the drift is caused by body-room temperature and saturation differences, which may vary from patient to patient according to the subject's pulmonary properties and breathing pattern. Such drifts may reach up to +/−3 Liters per minute in normals and in patients suffering from pulmonary disorders. Failure to correct for the drift may result in a substantial loss of accuracy which may render the result of the measurement as diagnostically irrelevant or even harmful in the case of miss-diagnosis.

The determination of reference volume levels is also useful in measuring minute by minute changes in Functional Residual Capacity (FRC) levels. This pulmonary property may be calculated by directly calculating changes in the absolute FRC level using, for example, the device described in International patent Application No. PCT/IL2008/001031 filed on Jul. 27, 2008 disclosing a system and method for determining FRC, TLC and RV. Alternately, minute by minute changes in FRC levels may be measured by continuously recording a volume time curve of a patient and identifying changes in the average level of FRC, as for example, the FRC line shown on FIG. 2. For example, a patient may be asked to breathe normally for several respiration cycles while the volume time curve is continuously recorded. An FRC level is then determined. Positive End Expiratory Pressure (PEEP) is then applied at the patient's mouth. The patient then breathes several respiration cycles until a new FRC level is determined. The change in FRC level due to the application of PEEP is given by the difference between the FRC level preceding and following the application of PEEP. In such a process, in addition to the drift described above, the continuous recording of exhaled and inhaled volume takes into account the contributions of the mechanism generating the PEEP and other possible contributions such as the continuous supply of oxygen.

Based on the reference pulmonary volumes, pulmonary volumetric data that is recorded during and/or after a respiratory modulation may be determined, for example as described in paragraphs [0047] through [0073] of International Patent Application No. PCT/IL00081/01031 filed on Jul. 27, 2008, which is incorporated herein by reference as if fully set forth herein. In such a manner, the volume at the selected respiratory instant may be identified.

Additionally, or alternatively, an instantaneous alveolar pressure, denoted herein as \tilde{P}_{Al}, of the user at the selected respiratory instant is provided as an instantaneous mechanical pulmonary property. Optionally, the instantaneous alveolar pressure is based on a reference alveolar pressure that is calculated during a respiratory modulation, such as a respiration interruption or an airway occlusion, for example as described in the International Patent Application No. PCT/IL00081/01031 filed on Jul. 27, 2008, which is incorporated herein by reference as if fully set forth herein. In such an embodiment, the reference alveolar pressure is equal to the airway pressure that is measured during an airway and alveolar equilibrium respiratory instant. That is to say, the reference alveolar pressure is the pressure measured or estimated in the airway and alveolar equilibrium respiratory instant.

Now, after the reference alveolar pressure is known, the dependence of P_{Al} on the volume at the specific respiratory instant may be determined. Optionally, P_{Al} is estimated and/or measured in several different respiratory stages, for example by interpolating known P_{Al} in known respiratory instants, for example as described in International Patent Application No. PCT/IL00081/01031 filed on Jul. 27, 2008, which is incorporated herein by reference. The continuous functional dependence of P_{Al} on lung volume under various breathing conditions may be interpolated, for example according to P_{Al} during FVC, SVC, IC and tidal breathing, before and after bronchodilators, in a supine or a seated pose.

Optionally, the instantaneous alveolar pressure and/or the pulmonary volume may be calculated using a breathing device, which may be a handheld device or a stationary device designed to be positioned at a physician desk, at a patient's bedside, and/or at the user premises.

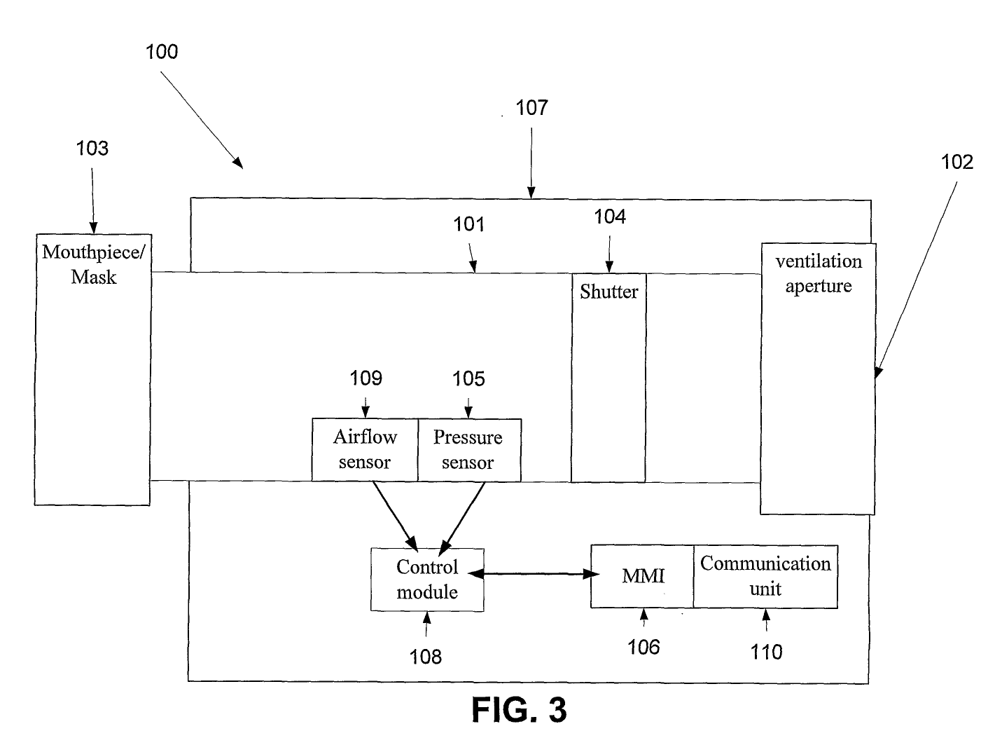

Reference is now also made to FIG. 3, which is a schematic illustration of an exemplary breathing device 100 for determining one or more instantaneous pulmonary measurements according to airway pressure and/or airflow which are measured at a breathing conduit thereof, according to some embodiments of the present invention. Optionally, the breathing device 100 comprises a breathing conduit, such as a chamber 101, optionally tubular, having a ventilation aperture 102, optionally in its end, and breathing end 103, such as a mouthpiece, and an endotracheal tube or a mask. Optionally the mask is designed for a human and/or veterinary use. In such an embodiment, the breathing device 100 may be adapted according to respective animalistic pulmonary properties. Optionally, the breathing end 103 is detachably secured to the chamber 101. Optionally, the breathing end 103 is disposable or easily cleaned and disinfected. Optionally, the breathing end 103 is connected to a mask that fits over the user's mouth and/or nose. Optionally, as shown at 104, a shutter is constructed in the chamber 101, for example in a plane that is perpendicular to an axis between the ventilation end 102 and the mouthpiece end 103. In such a manner, the shutter 104 may be used for occluding the airway during an airway occlusion event, for example as described in International Patent Application No. PCT/IL00081/01031 filed on Jul. 27, 2008 which is incorporated herein by reference. The occluding shutter 104 allows the breathing device 100 to regulate the passage of air flux in the chamber 103. The shutter 104 is connected to a control module 108 that is designed to control the opening and/or closing of the shutter 104. Optionally, the shutter 104 is designed to generate a relatively small compression wave during the occlusion.

The breathing device 100 comprises one or more sensors for analyzing the airflow and/or pressure in the chamber. For example, as shown at 105, a pressure sensor is positioned between the shutter 104 and the mouthpiece end 103. The pressure sensor 105 may be any pressure measurement component, such as manometer or sensor for the measurement of absolute pressure with an analog to digital sampling rate of 100 Hz, 1000 Hz, 5000 Hz, 10,000 Hz or any intermediate value or larger value. Such pressure sensors are readily available and may be acquired, for example, from Honeywell Catalog #40PC001B1A, which is incorporated herein by reference. One example of such a sensor is a Samba 3000 pressure transducer, which is available from Linton Instrumentation of Norfolk, England. Other pressure measuring sensors may be used as the application requires. The pressure sensor 105 may be fabricated for example from a respiratory airflow resistive means and a differential pressure manometer, or alternatively from a Pitot tube and a differential pressure manometer. The differential pressure manometer may be any suitable sensor with a data rate of 100 Hz, 1000 Hz, 5000 Hz and 10,000 Hz or any intermediate value or larger value. Such differential pressure manometers are readily available, for example in Honeywell Catalog #DC002NDR4, which is incorporated herein by reference.

Optionally, as shown at 109, a flow sensor, such as a mass respiratory airflow sensor, is positioned in the chamber 110, for example between the shutter 104 and the mouthpiece end 103. The flow sensor may be any flow sensor, such as a hot wire mass respiratory airflow sensor and a mass respiratory airflow (MAF) sensor.

Optionally, the chamber 103 is constructed in a housing 107 generally sized and shaped so that it can be held comfortably in one hand and held-up to a user's mouth while measuring the pulmonary alveolar pressure of the user, for example as outlined above and described below. The breathing device 100 may also be configured so that the user can comfortably hold the breathing device 100 and self monitor their respiratory parameters and/or self operate the device and/or manage a PFT.

Optionally, the breathing device 100 comprises a man machine interface (MMI) 106, such as a control panel, for example a keypad, a touch screen, and a set of buttons and a liquid crystal display (LCD) screen. The keypad may include a start button, a selection button or other controls as desired to operate the breathing device 100, and to facilitate measurement of pulmonary alveolar pressure of the user and/or triggering shutter events, for example as described below. The airflow and pressure sensors 109, 105 may be in communication with the control module 108 so that measured pressure is transferred to thereto. The control module 108 may be connected to a memory (not shown) to store the measured pressure, flow rate and/or the calculations which are based thereon.

The control module 108 is optionally designed for receiving the outputs of the pressure sensor 105 and/or a respiratory airflow sensor 109 to calculate the instantaneous pulmonary alveolar pressure and the instantaneous pulmonary volume, for example as described in International Patent Application No. PCT/IL00081/01031 filed on Jul. 27, 2008 which is incorporated herein by reference. The outputs of the control module 108 may be presented on the MMI.

Reference is now made, once again, to FIG. 1. As described above, a plurality of instantaneous mechanical pulmonary properties, such as instantaneous alveolar pressure and/or pulmonary volume may be provided or measured, for example as described above in relation to 201. Then, as shown at 202, the plurality of instantaneous mechanical pulmonary properties may be correlated. As used herein a correlation may refer to an association between instantaneous mechanical pulmonary properties of a user which are measured at the same respiratory instant and/or stage, a mapping and/or binning instantaneous mechanical pulmonary properties of a user which are measured at the same respiratory instant and/or stage, and/or a scaling and/or normalizing one instantaneous mechanical pulmonary property of a user according to another instantaneous mechanical pulmonary property which is measured at the same respiratory instant and/or stage. For clarity, the same respiratory instant or stage may be the same instant in different respiration maneuvers and/or a unique respiratory instant or stage in a unique respiration instant. For example, the aforementioned instantaneous pulmonary volume in a selected respiratory instant may be correlated to instantaneous alveolar pressure in the selected respiratory instant. According to other examples, the respiratory system compliance, airway resistance, alveolar pressure and derivatives thereof may be correlated with minute by minute changes in the FRC level, as described above. Other correlations, for example between the aforementioned instantaneous pulmonary volume and various pulmonary functioning curves may also be performed. Optionally, the correlating allows adjusting the pulmonary volume according to the estimated alveolar pressure that is applied on the pulmonary tissues in the selected respiratory instant, thereby improving the diagnostical value thereof. Optionally, the correlating allows adjusting the externally applied positive end expiratory pressure to trigger changing the pulmonary volume, for example, the FRC level, thereby improving the pulmonary functioning of the patient. Optionally, the correlating allows monitoring the pulmonary functioning of intubated and/or non-invasively ventilated patients. Such monitoring helps attending physicians to assess the response of patients to extubation and/or the probability of successful weaning from invasive and/or non-invasive mechanical ventilation. Optionally, the correlating allows monitoring pulmonary functioning of patients after extubation. Optionally, an instantaneous cumulative value that is based on the pulmonary volume in the light of the instantaneous alveolar pressure, such as a ratio between them that may be calculated.

As shown at 203, the correlated instantaneous mechanical pulmonary properties allow for determining one or more instantaneous pulmonary measurements, such as respiratory system compliance and a respiratory system compliance index during tidal breathing in respective respiratory instants, for example as described below. Optionally, as shown at 204, 201-203 are repeated a plurality of times for measuring the instantaneous pulmonary measurements in a plurality of respiratory instants during one or more respiration cycles of the user. In such a manner, the method may be used for generating an analysis of the pulmonary property along various stages of a PFT. Additionally or alternatively, the method may be used for monitoring the breathing of the user by providing a real time indication of the clinical status of the user in various respiratory instants. As used herein, real time or substantially immediately might refer to presenting, forwarding and/or otherwise providing instantaneous pulmonary properties such that there is no significant, noticeable, and/or inconvenient delay experienced by the user and/or a receiving client terminal. For example, real time may be less than 30, 20, 10, 5, 1, 0.1, 0.01, 0.001 seconds, and/or any intermediate value.

As described above, the method may be implemented using the breathing device 100. The breathing device's control module 108 may be used for performing calculations based on the outputs of the aforementioned sensors. Optionally, the breathing device 100 is a handheld device 100 that allows the physician to perform various PFTs, for example as described below.

Optionally, the outputs of the aforementioned sensors and/or the outputs of the control module 108 may be forwarded, for example by using a communication unit 110. Optionally, the communication unit 110 comprises a short-range radio interface, such as a Bluetooth™ transceiver, which is defined according to IEEE 802.15.1 specification that is incorporated herein by reference, optionally utilizing a Bluetooth™ enhanced data rate (EDR) chip that is defined according to Bluetooth™ core specification version 2.0+EDR of the Bluetooth™ special interest group (SIG), which is incorporated herein by reference, or a Wibree® transceiver. Optionally, the communication unit 110 comprises a radio transceiver that uses ultra-wideband (UWB) frequencies. In such an embodiment, the wireless interface may establish a wireless personal area network (WPAN) according to WiMedia™ specification or according to the Wireless USB (WUSB), which are incorporated herein by reference. Optionally, the communication unit 110 supports serial port profile (SPP) connections between the monitoring device 100 and the remote server and/or management unit. Optionally, the communication unit 110 comprises a wireless local area network (WLAN) interface such as a radio transceiver that uses high frequency radio signals, which are defined according to a WLAN standard, for example 802.11a, 802.11b, 802.11g, and 802.11n standards, which are herein incorporated by reference. The WLAN interface optionally uses high frequency radio signals. In such an embodiment, the WLAN interface is defined according to WiMAX™ IEEE 802.16 standard or wireless FireWire IEEE 802.15.3 standard, which are incorporated herein by reference.

Optionally, the communication unit 110 is designed to communicate with a client terminal, such as a personal digital assistant (PDA), a Smartphone, a thin client, a laptop, a wrist watch, and/or any other terminal which may be used for receiving the outputs of the sensors and/or the control module. Such a client terminal may be used to perform the calculations and/or methods described herein and/or to present the data that is calculated by the control module 108.

Optionally, the handheld device 100 is designed to allow a patient to perform self measurement and/or diagnosis of the PFTs. In such an embodiment, the MMI 106 may be used to instruct the user during the process and/or to present recommendations and/or alerts to the patient. Optionally, the communication unit 110 forwards the calculation to the client terminal and/or to a remote monitoring center that allows the recording thereof and/or alerts the user, the user's physician and/or any other intensive care system accordingly. Optionally, the size of the device allows the physician and/or the user to carry it from one place to another in a commonly used bag, such as a handbag.

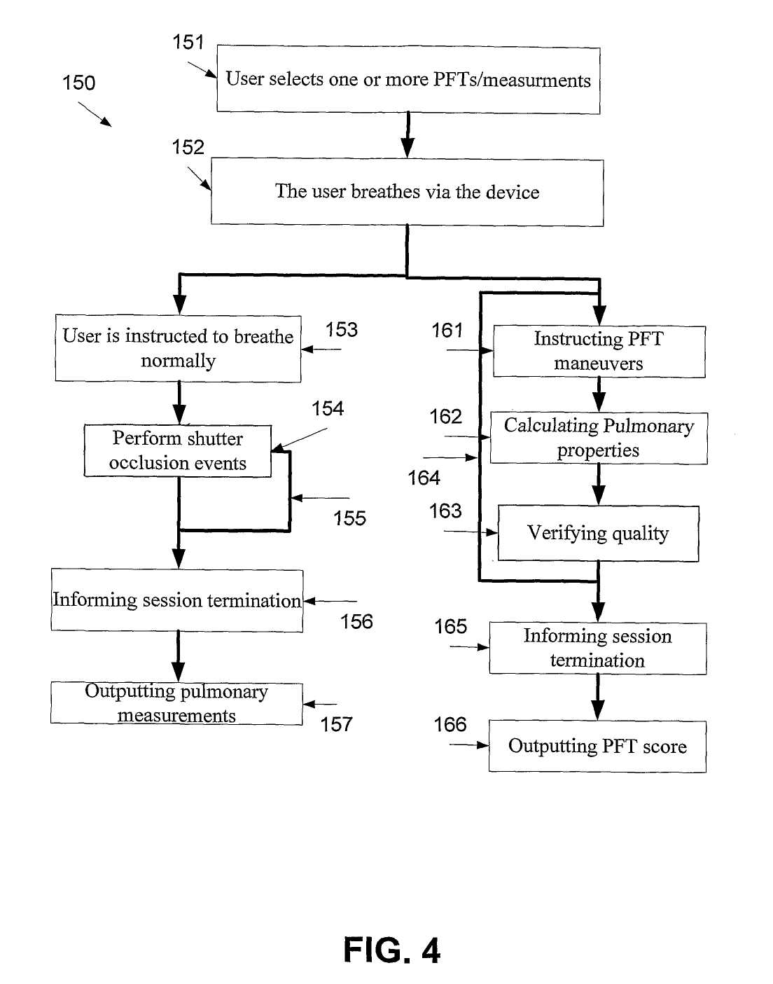

Reference is now made to FIG. 4, which is a flowchart 150 of a method for allowing a user to operate the breathing device for performing selected PFTs, according to some embodiments of the present invention. By implementing the method, the user may normally breathe into the breathing device 100 a certain number of cycles until enough data is collected and then the user is issued an output that includes pulmonary measurements, optionally instantaneous, such as respiratory system compliance index during tidal breathing, airway resistance, TGV, and any combination thereof. The method may allow causing the user to follow PFT maneuvers.

As shown at 151, a respiration analysis session is initiated when the user uses the MMI 106 to input one or more selected PFTs and/or measurement detection and/or to request the initiation or continuation of a PFT or a series of PFTs. Now, as shown at 152, the user breathes via the device. Optionally, as shown at 153, the user breathes normally. Then, as shown at 154 one or more shutter occlusion events are performed, for example as described in International Patent Application No. PCT/IL00081/01031 filed on Jul. 27, 2008, which is incorporated herein by reference. Optionally, the one or more shutter occlusion events are performed after a normal breathing is detected. A shutter occlusion event allows calculating one or more pulmonary properties, for example as described in International Patent Application No. PCT/IL00081/01031 filed on Jul. 27, 2008, which is incorporated herein by reference. Optionally, as shown at 155, the one or more shutter occlusion events are repeated until a requested number of pulmonary property estimations is performed. As shown at 156, the user is informed that the respiration analysis session has been terminated. Then, as shown at 157, one or more pulmonary measurements, such as a respiratory system compliance index during tidal breathing, an airway resistance, a TGV and any correlation or combination thereof, for example with other processes and/or data are presented to the user.

As shown at 161, the user may be caused to perform PFT maneuvers. The user performances are optionally recorded, for example as described above. During the PFT maneuvers, as shown at 162, the user pulmonary properties are calculated, for example as described above. Optionally, the quality of the PFT maneuvers is verified, as shown at 163. In such an embodiment, as shown at 164, 161-163 may be repeated a plurality of times until a desired quality is acquired. As shown at 165, the user is informed that the respiration analysis session has been terminated. Then, as shown at 166, the outcome of the PFT, which may include respiratory system compliance indices, an airway resistance, a TGV, a respiratory muscle endurance or strength test, spirometry parameters and any correlation or combination thereof, is presented to the user, for example using the MMI 106.

Reference is now made, once again, also to FIG. 1. According to some embodiments of the present invention, the one or more instantaneous pulmonary measurements include a respiratory system compliance estimation. For clarity, as the respiratory system compliance is the reciprocal of lung elasticity, respiratory system compliance may be understood herein as inverse respiratory system elasticity. Optionally, in use, the calculation is by the control module 108 based on the outputs of the aforementioned sensors.

Optionally, the aforementioned cumulative value is a ratio that is calculated as follows:

$$\frac{d V}{d {\tilde{P}}_{Al}}$$

where \tilde{P}_{Al}, as outlined above, denotes an alveolar pressure in a selected respiratory instant, referred to herein as an instantaneous alveolar pressure, during a continuous breathing. Accordingly, the outputted respiratory system compliance is calculated using \tilde{P}_{Al} as follows:

$${\tilde{C}}_{L} = \frac{d V}{d {\tilde{P}}_{Al}}$$

where \tilde{C}_{L} denotes compliances obtained during expiration and/or inspiration respiratory instants during one or more respiration cycles.

In some embodiments of the present invention, the instantaneous alveolar pressure and the pulmonary volume are used for calculating the respiratory system compliance during tidal breathing, referred to herein as a tidal compliance.

As commonly known, during tidal expiration, air is expelled from the lungs primarily due to the elastic contraction of the alveoli. Therefore, respiratory system compliance measured during relaxed tidal breathing is indicative of the elasticity level of the alveoli. In addition, the functional dependence of the lung compliance, as opposed to respiratory system compliance, on volume between the V=0 and tidal breathing levels is approximately linear. Alternately, it can be assumed that when the lungs are fully deflated, the pressure at the empty lungs is equal to the ambient pressure. Thus, by assuming V_{b}, P_{b}=0, from the equation presented in the background section, a tidal compliance index may be calculated as follows:

$${C}_{Tidal} = \frac{{V}_{0}}{{\tilde{P}}_{Al}}$$

where V₀ denotes the volume level at which C_{Tidal} is measured.

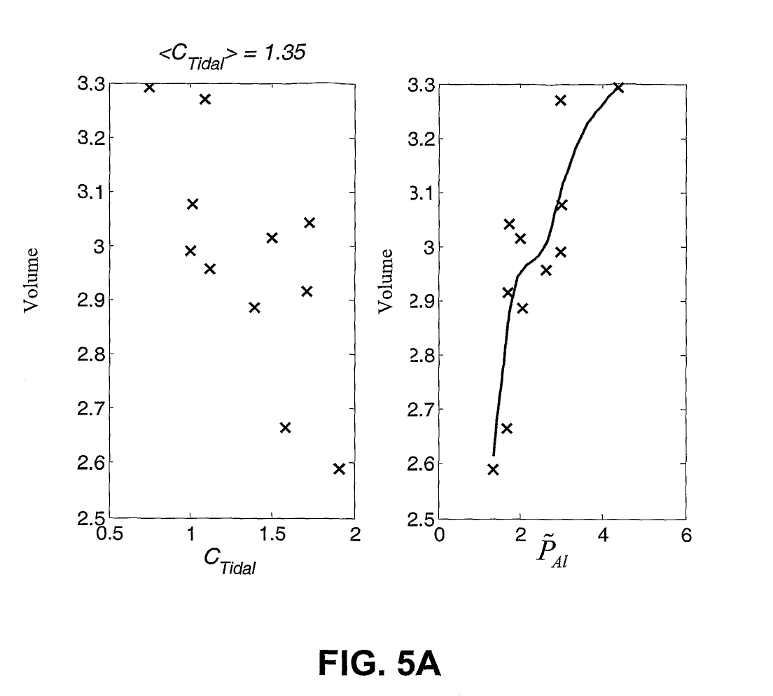

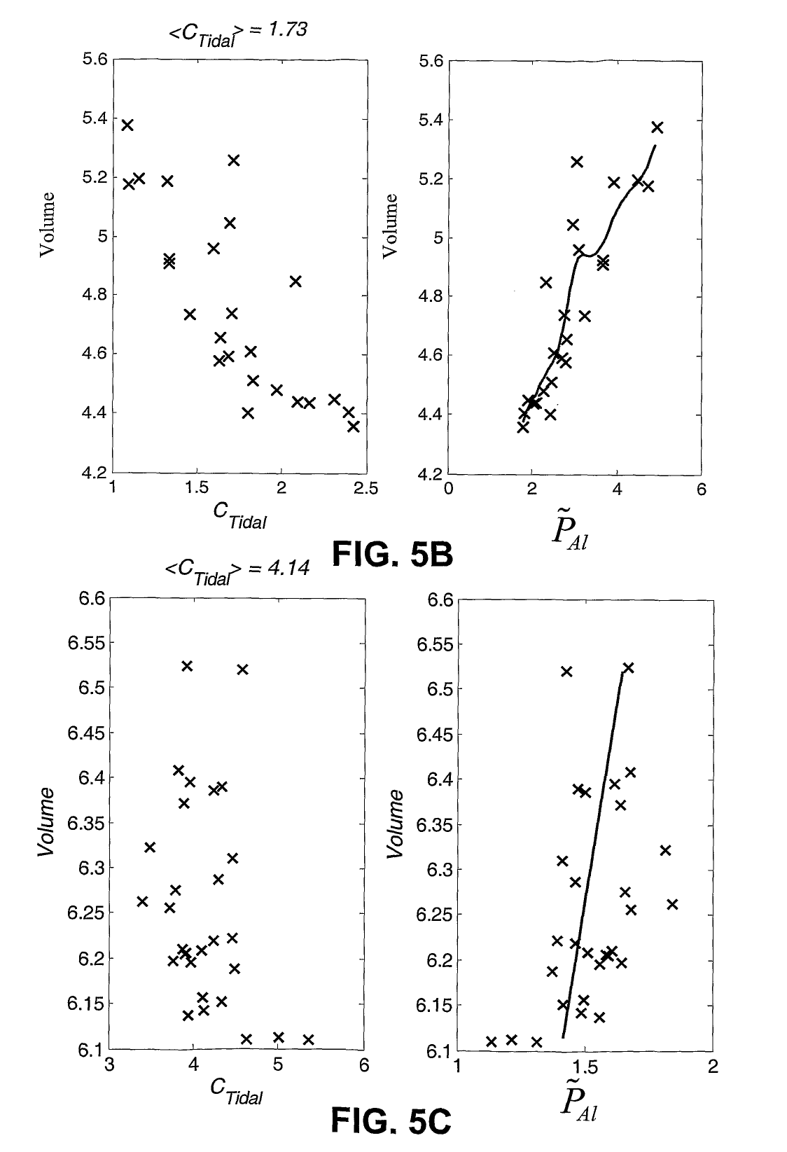

C_{Tidal} may be used for diagnosing the pulmonary functioning of the user. For example, reference is now also made to FIGS. 5A-5C which are graphs depicting exemplary instantaneous pulmonary volumes, plotted against expiratory C_{Tidal} and a respective P_{Al} in a respective respiratory instant. FIG. 5A data pertains to a healthy user, FIG. 5B data pertains to a user having mild emphysema with forced expiratory volume—one second (FEV1) of 56%, and FIG. 5C data pertains to a user having severe emphysema with FEV1 of 23%. All the users are around 40 years of age and have a TLC of about 7 L. The mean C_{Tidal} as well as the dependence of V₀ on \tilde{P}_{Al} in these examples vary with the severity of the emphysema. The instantaneous volume is plotted against \tilde{P}_{Al} and a smoothed interpolation thereof at V₀(\tilde{P}_{Al}) is depicted by a solid line.

As commonly known, inspiratory muscles are contracted to inspire air into the lungs during tidal inspiration. Thus, C_{Tidal} varies during expiration and inspiration for similar V₀, depending on the state of the alveolar tissue and inspiratory muscles.

For clarity, C_{T} denotes a ratio between expiratory C_{Tidal} and inspiratory C_{Tidal}, [symbol] c_{T} [symbol] denotes a ratio between the average of inspiratory C_{Tidal} and the average of expiratory c_{Tidal}. In healthy users [symbol] c_{T} [symbol] →1 and in patients with higher than normal alveolar tissue compliance [symbol] c_{T} [symbol] →0. In some patients suffering from fibrotic lung conditions, [symbol] c_{T} [symbol] >1. Thus, [symbol] c_{T} [symbol] may be used for determining whether the alveolar tissue compliance is higher, lower, or equal to a normal level.

It should be noted that C_{Tidal} and [symbol] c_{T} [symbol] may be based on data gathered during normal, relaxed breathing, for example using the method depicted in FIG. 2. As such, C_{Tidal} and [symbol] c_{T} [symbol] may be calculated with minimal patient cooperation during the respiratory data gathering. In addition, C_{Tidal} is calculated using a single interruption and is therefore indicative of the respiratory system compliance at the instant it is being measured. This differs from respiratory system compliance calculations which depend on two points along the pressure volume curve as in these calculations the natural state of the respiratory system may be affected by the interruption. C_{Tidal} is therefore more adapted for minute by minute monitoring of the respiratory system compliance.

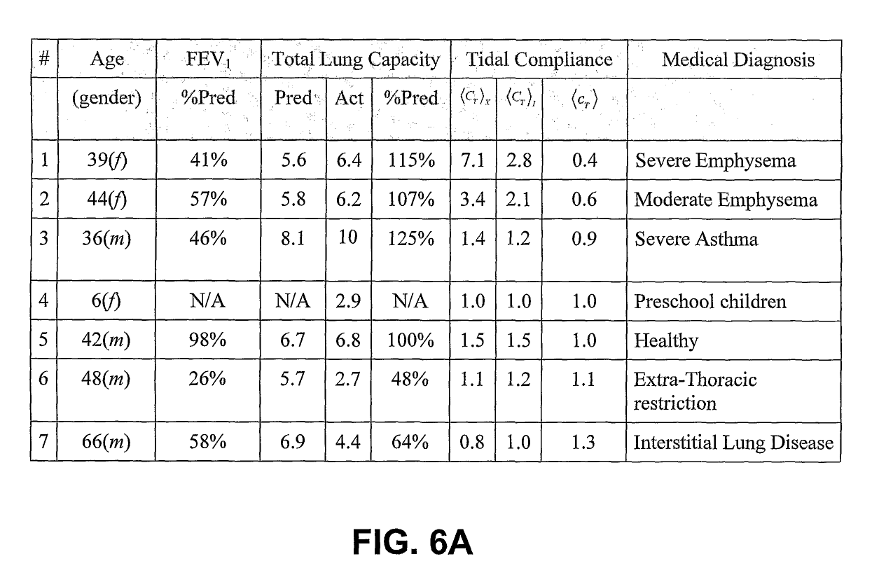

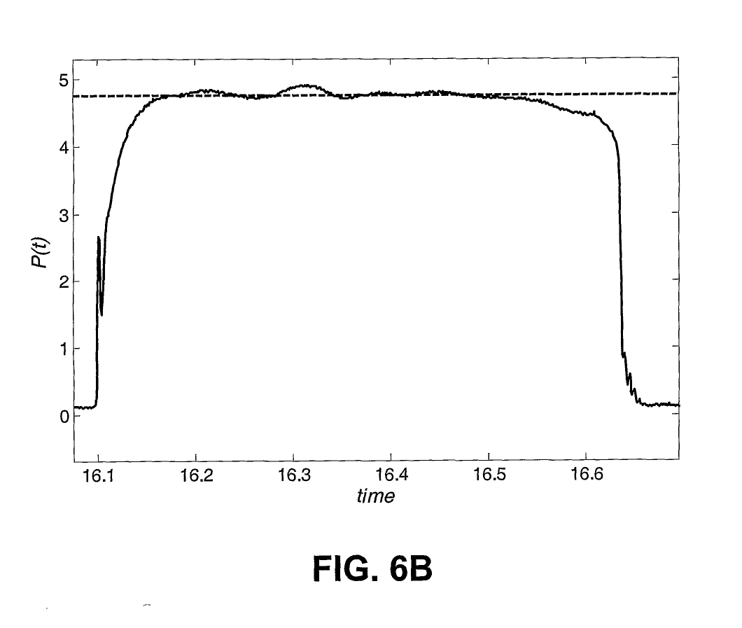

FIG. 6A depicts a table that lists expiratory and inspiratory averaged C_{Tidal}, denoted herein as [symbol] c_{T} [symbol] _{X} and [symbol] c_{T} [symbol] _{I}, and [symbol] c_{T} [symbol] of users of varying ages and medical conditions. For clarity, the units used are Liters for volume measurement and mmHg for pressure. The age, gender, TLC and medical diagnosis are also given. As shown in FIG. 6A, the inspiratory and expiratory compliance of users with emphysema increases with the severity of the disorder, ranging between 2.0 and 10.0 while the ratio between the Expiratory and Inspiratory tidal compliance decreases with the severity of the disorder. The Inspiratory and Expiratory compliance and the ratio between the Expiratory and Inspiratory compliances of users with asthma are in the same range as healthy users while the TLC and FEV1 resemble those of users with emphysema. The Expiratory and Inspiratory compliances of users with restrictive disorders, such as interstitial lung disease (ILD) and Extra Thoracic restriction tend to be in the same range or below those of healthy users while the ratio between the expiratory and inspiratory compliances tends to be close to or higher than 1.

Optionally, [symbol] c_{T} [symbol] _{X}, [symbol] c_{T} [symbol] _{I} and [symbol] c_{T} [symbol] may be used to differentiate users suffering from Emphysema from users suffering from Asthma. As commonly known, volumetric and airway resistance measurements of users with Asthma may be similar to those of users with Emphysema. Therefore, a diagnosis of Asthma or Emphysema cannot be based on volumetric or airway resistance measurements only. Optionally, the calculation of [symbol] c_{T} [symbol] _{X}, [symbol] c_{T} [symbol] _{I} and [symbol] c_{T} [symbol] may be used for indicating the absence of Emphysema in patients with Asthma, see Todd M., Riccardo Pellegrino, Vito Brusasco, and Joseph R. Rodarte. Measurement of pulmonary resistance and dynamic compliance with airway obstruction. J. Appl. Physiol. 85(5): 1982-1988, 1998, Harris RS Pressure-volume curves of the respiratory system, Respir Care. 2005 January; 50(1):78-98, which are incorporated herein by reference.

It should be noted that the definitions and method of compliance calculated under tidal respiratory conditions are not restricted to tidal respiratory conditions and may apply to a plurality of respiratory states such as, for example, in the vicinity of the TLC or RV levels, forced or static respiratory states, and supine or seated respiratory positions. For example, optionally, [symbol] c_{T} [symbol] _{I} or a similar respiratory system compliance index during forced breathing can be used to access the inspiratory muscle strength and endurance. Such measurements or continuous monitoring may assist in predicting weaning success of mechanically ventilated patients.

Reference is now made to FIG. 6B, which is an exemplary graph of an exemplary pressure curve shown as a solid line and an averaged pressure level, shown as a dashed line during an airflow interruption, according to some embodiments of the present invention.

The dashed line denotes the averaged pressure level once the airflow pressure has reached a static uniform state. The duration of the occlusion event shown on FIG. 6B is 500 ms which is sufficient in the shown example for the lungs to achieve a uniform pressure level, although the duration it takes different users, during different respiratory conditions and different embodiments of the device to reach a static pressure level may be longer or shorter than 500 ms. The uniform pressure level may be considered to reflect the alveolar pressure during static lung conditions. Optionally, the volume level at which airways are occluded and static pressure is measured is controlled or aided by an external ventilation apparatus which inflates or deflates the lungs. By using the pressure thus obtained, an index of the static respiratory system compliance may be calculated as described above, denoted as C_{S}, or the averaged value over more than one occlusion event, denoted as [symbol] c_{S} [symbol] , in a manner similar to [symbol] c_{T} [symbol] _{X} and [symbol] c_{T} [symbol] _{I} using the method for correlating pulmonary characteristics according to alveolar pressure and/or lung volume, for example as described in International patent Application No. PCT/IL2008/001031 filed on Jul. 27, 2008, which is incorporated herein by reference.

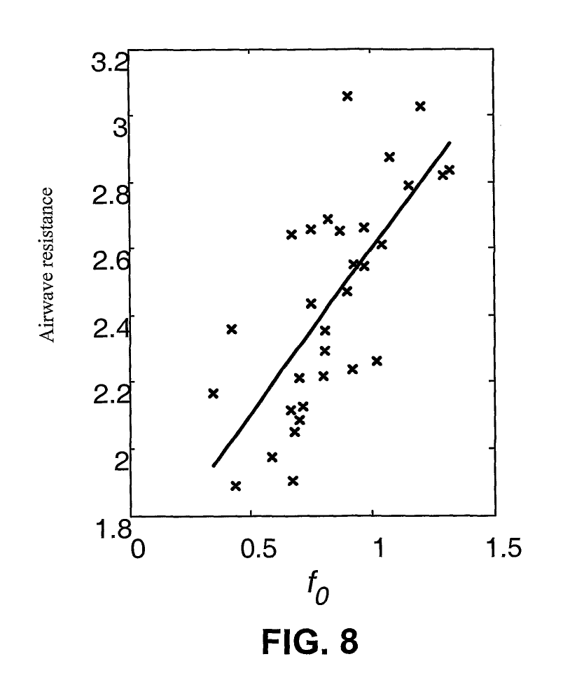

According to some embodiments of the present invention, the one or more instantaneous pulmonary measurements include a pulmonary airway resistance, denoted as R_{aw} and optionally instantaneous pulmonary volume and/or alveolar pressure. According to Poisseuile's law the airway resistance is approximately proportional to the airway's length and approximately inversely proportional to some power of the airway's caliber. During respiration, the alveolar pressure and the airway's length and caliber depend on the volume of the lung. Therefore, the pulmonary airway resistance is related to the lung volume level at which it is measured. For example, airway resistances, which are measured at equal airflow rates close to the FRC level are generally observed to be larger than close to the TLC level, due to differences in the airway's cumulative cross-sectional area and alveolar pressure, see Kochi T, Bates J H, Okubo S, Petersen E S, and Milic-Emili J: Respiratory mechanics determined by flow interruption during passive expiration in cats. Respir Physiol. 1989 November; 78(2):243-52, which is incorporated herein by reference. Thus, correlating the airway resistance in a certain instant during a respiration cycle according to a respective alveolar pressure or volume level may improve the diagnostic relevance of measured airway resistance. In addition, airway resistance may strongly depend on the degree of turbulence in the flow, which is itself dependent on the flow. For example, FIG. 8 depicts a dependence of airway resistance R_{aw} on the instantaneous flow, denoted herein as f₀, which is calculated using the breathing device 100. R_{aw} exhibits a clear increasing trend with f₀. Thus, since R_{aw} depends on the flow rate and lung volume, the correlation thereof with respective measurements such as, RV, TGV and/or TLC, allows for generating a more accurate estimation of the actual airways resistance of the user during different stages of a respiration cycle.

Optionally, the pulmonary airway resistance, denoted herein as R_{aw}, may be defined as follows:

$${R}_{aw} = \frac{{P}^{′} - P}{f}$$

where f denotes the respiratory airflow, as described above, and P′ and P denote the pressure at the two ends of an airway segment. Optionally, the airway resistance is calculated using the breathing device 100 by setting P′ as the alveolar pressure P_{Al}, P as the pressure at the airway, for example as estimated in the mouth, Pₘ, and f as the flow at the mouth f₀ during an instant of a shutter occlusion, see Von Neergaard K, Wirz K. Die Messung der Strömungswiederstände in den Atemwegen des Menschen, insbesondere bei Asthma and Emphysem. Z Klin Med 1927; 105: 51-82, which is incorporated herein by reference. Airway resistance may aid in the diagnosis of patients suffering from flow limitation by assessing the degree of flow limitation and the response of airways to bronchial challenge tests, drugs, and/or other types of medical intervention. For example, pulmonary airway resistance is expected to decrease significantly in patients suffering from Asthma after the administration of bronchodilators. The response of up to 25% of patients suffering from Emphysema to bronchodilators may be similar to that experienced by patients suffering from Asthma.

In some embodiments of the present invention, the one or more instantaneous pulmonary properties include instantaneous pulmonary data which is measured during the PFT and the aforementioned instantaneous pulmonary volume. A correlation of these instantaneous pulmonary properties allows the physician to diagnose the pulmonary or cardiac fitness of the user according to her performances in various respiratory stages. Optionally, the breathing device 100 may be used for continuously monitoring the instantaneous pulmonary data. For example, the device may be used for calculating one or more instantaneous pulmonary properties while the user performs an exertion test, such as an ergometry test or a cardio-pulmonary exercise test (CPX).

In such embodiments, instantaneous pulmonary measurements, such as the aforementioned tidal compliance may be continuously calculated during a PFT, for example as described above in relation to FIG. 1. These instantaneous pulmonary properties may be presented in real time, during the PFT, allowing the physician to diagnose patients based on a combination of their performances in various PFT stages and instantaneous mechanical pulmonary properties, such as current pulmonary volume and/or alveolar pressure.

For example, the method may be used to estimate instantaneous pulmonary measurements during PFTs, such as spirometry and cardio and/or respiratory tests, tests of respiratory muscle endurance, Cardio-Pulmonary Exercise tests, and Respiratory Muscles Strength tests which could be used in the evaluation of, for example a patient suffering from chronic obstructive pulmonary disease (COPD). For another example, the method may be used for estimating instantaneous pulmonary properties of a patient being weaned from invasive or non-invasive mechanical ventilation.

According to some embodiments of the present invention, the PFT is spirometry, such as forced expiratory volume in 1 second (FEV1). In such an embodiment, the airflow sensor 109, which is optionally a pneumotachometer, measures the amount of air and the rate of air that is breathed in and out over a specified period and the control module is optionally designed to generate a volume-time curve and/or flow-volume loop based thereupon, for example as commonly known in the art. Optionally, the spirometry is performed as defined by the American Thoracic Society (ATS) and the European Respiratory Society (ERS), which the protocols thereof are incorporated herein by reference. From this volume-time curve and/or flow-volume loop, vital capacity, tidal volume, breathing rate and ventilation rate may be induced. In parallel to the volume-time curve and/or flow-volume loop, the pressure and/or the airflow sensors may detect airflow and pressure in the chamber, allowing the calculation of instantaneous alveolar pressure, instantaneous volume and/or additional instantaneous pulmonary measurements which are based on the flow rate measured before, during, and or after the spirometry, for example as described in International patent Application No. PCT/IL2008/001031 filed on Jul. 27, 2008, which is incorporated herein by reference.

In such an embodiment, each respiratory instant in the volume-time curve and/or flow-volume loop may be associated with a respective instantaneous alveolar pressure and/or other instantaneous pulmonary properties of the lung in a respective respiratory instant. In such a manner, the physician can provide a more accurate spirometry diagnosis which is based on the various stages of the user respiration.

Optionally, some or all of the respiratory instants which are presented in the volume-time curve and/or flow-volume loop are associated with respective instantaneous tidal compliance, measured or estimated for respective respiratory instants, optionally calculated as described above.

For clarity, a spirometry test, for example as described above, provides useful reproducible outcomes which are indicative of an airflow obstruction. For example, volume-time curve and/or the flow-volume loop indicate the presence of airway obstruction. However, this indication does not teach the origin of the medical condition, for example whether Asthma, COPD, Emphysema, and/or any other pathological symptom or indication. The aforementioned association between instantaneous respiratory parameters, such as a pulmonary volume, tidal compliance and/or tidal elasticity provides a comprehensive evaluation of lung mechanics and allows the physician to diagnose the origin of the airway obstruction. For example, tidal compliance measurements would identify the loss of lung elastic recoil and hyperinflation in near-fatal Asthma patient, see Gelb A F, Schein A, Nussbaum E, et al. Risk factors for near-fatal asthma, Chest 2004 October; 126(4):1138-46, which is incorporated herein by reference.

According to another example, the respiratory system compliance is measured in combination with an airway resistance measurement. Such a combination may be used for diagnosing and/or estimating an ability of a patient to sustain autonomous breathing while being supported by ventilation or following weaning from ventilation support for diagnosing weaning success probability.

According to another example, the respiratory system compliance is measured in combination with Work Of Breathing (WOB). The WOB is composed of the work required to expand the lungs against its elastic recoil and the work required to maintain airflow against the airway resistance. The WOB can be calculated if the respiratory system resistance and compliance are known or directly from the respiratory system volume-pressure relation. The WOB may assist in predicting weaning success from ventilation support and in monitoring the need for ventilation support.

According to another example, the respiratory system compliance is measured in combination with respiration rate, the number of respiratory cycles per minute.

According to another example, the respiratory system compliance is measured in combination with ventilation rate. The ventilation rate is the product of the respiration rate and the average breathing volume. For example, during tidal breathing, the ventilation rate is equal to the product of the tidal respiration rate and the tidal volume. Abnormal respiration rates or ventilation rates may be indicative of lung or cardiac disease, central nervous disease, or metabolic disorders and need to be considered when predicting weaning success from ventilation support and in monitoring the need for ventilation support.

According to another example, the respiratory system compliance is measured in combination with is minute by minute changes in Functional Residual Capacity (FRC) levels, as described above. Measurement of minute by minute changes in FRC level in combination with respiratory system compliance may assist in determining optimal PEEP levels and other parameters of ventilation support, sudden respiratory stress and stability of the respiratory system.

According to another example, the respiratory system compliance is measured in combination with Expiratory Flow Limitation (EFL). One of the methods used for detecting EFL is the application of Negative Expiratory Pressure (NEP). As commonly known, NEP may be used for detecting EFL by generating a NEP test event, which may be understood herein as an artificial reduction of pressure at the airway which induces an increase in the intrathoracic pressure gradient. The EFL is detected when the reduction of pressure at the airway does not consecutively increase the airflow that is measured in the airway.

The existence and severity of EFL (including upper airway obstruction) is closely related to the level of airway obstruction. One way of measuring airway obstruction is by measuring the air exhaled during the first second of forced expiration from the level of TLC. The level of airway obstruction during relaxed breathing may also be estimated by the interval it takes for the pressure at the mouth to equilibrate with the alveolar pressure and the difference between the pressure at the mouth and the alveolar pressure. Such an estimation of the level of obstruction may also depend on the level of airflow at the instant of interruption and on the measuring respiratory instant during the respiration cycle.

Additionally or alternatively, the method may be used for estimating and correlating instantaneous pulmonary properties during a negative expiratory pressure (NEP) test, see, Calverley P M A, Koulouris N G (2004) Flow limitation and dynamic hyperinflation: key concepts in modern respiratory physiology. Eur Respir J 25:186-199, which is incorporated herein by reference. The breathing device 100 may be adjusted to induce the aforementioned artificial reduction of mouth pressure and for detecting the absence or presence of the consecutive increase of airflow for allowing the performance of the NEP test. NEP has been shown to be useful in detecting EFL in adult cystic fibrosis patients, see, Holland A E et al., Does expiratory flow limitation predict chronic dyspnoea in adults with cystic fibrosis?, Eur Respir J 28: 96-101. 2006, exercising asthmatics, see, N. Kosmas, J. Milic-Emili, A. Polychronaki, I. Dimitroulis, S. Retsou, M. Gaga, A. Koutsoukou, Ch. Roussos, and N. G. Koulouris Exercise-induced flow limitation, dynamic hyperinflation and exercise capacity in patients with bronchial asthma Eur. Respir. J., Sep. 1, 2004; 24(3): 378-384. and sleep apnea, see Van Meerhaeghe A et al., Operating characteristics of the negative expiratory pressure technique in predicting obstructive sleep Apnoea syndrome in snoring patients, Thorax 2004; 59; 883-888. All of these reference are incorporated herein by reference.

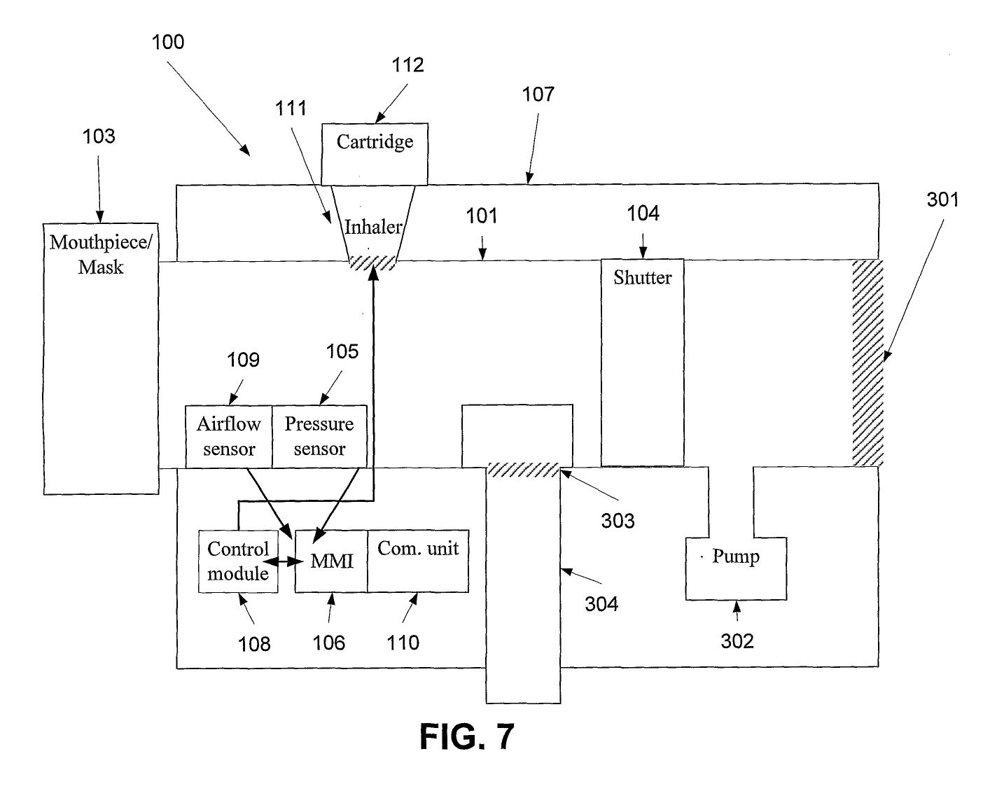

Reference is now made to FIG. 7, which is a schematic illustration of the breathing device 100 for performing a rapid injection or extraction of air, also suitable for a NEP test, while measuring instantaneous alveolar pressure and/or volume, according to some embodiments of the present invention. The sensors 105, 109, the MMI 106, the communication unit 110, the shutter 104, the control module 108 and the mouthpiece/mask 103 are as described above in relation to FIG. 2. However, FIG. 7, further depicts a closable flow tube 304 having a valve 303 that is connected to the chamber 101 between the shutter and the mouthpiece 103, a ventilation valve 301 that is positioned in the backend of the chamber 101, and a pump 302 that is connected to the chamber between the shutter 104 and the ventilation valve 301. Optionally, the pump is designed to propel away from the chamber, thereby to induce expiration or resist inspiration. Optionally, the pump is designed to propel air into the chamber, and thereby induce inspiration or resist expiration. Optionally, the closable flow tube 304 is a T shaped tube that allows the airflow in the breathing device to bypass the shutter 104. Optionally, the closable flow tube 304 is a T shaped tube that directs airflow into a sealed container. The closable flow tube 304 may be close, automatically and/or manually at one or more velocities. Optionally, the pump 302 is located between the mouth of the user and the shutter 104. Optionally, the shutter 104 is located between the mouth of the user and the pump 302. Optionally, the chamber 101 is connected to an external container of varying volumetric capacity. Optionally, the pump 302 injects or extracts air from the portion at a rate that is significantly faster than the airflow rate of respiration at the instant of measurement.

In use, a NEP test event is initiated when the user expires through the mouthpiece 103 while the closable flow tube 304 is in an open configuration and the shutter is occluded. Then, the pump 302 propels air to reduce the pressure between the closed shutter 104 and the ventilation valve 301 which is optionally in a closed configuration. This propelling does not affect the air pressure between the closed shutter 104 and the mouthpiece 103. Optionally, the reduced pressure is controlled by the ventilation valve 301. After the pressure has been reduced to and/or below a requested level, for example 1 mmHg, 5 mmHg, 10 mmHg, 50 mmHg, or any intermediate value or larger value below the pressure level at the mouth, the shutter 104 is reopened. Optionally, the reopening velocity is determined according to the requirements of the testing. As a result, the pressure between the shutter 104 and the mouthpiece 103 is reduced in a manner that forces expiration. The forced expiration facilitates EFL detection by recording the effect the pressure reduction pressure on the airflow that is measured by the airflow sensor 109. An indication of the existence of EFL is provided when the response of the airflow recorded at the airways is not proportional to the increase in the pressure gradient due to the reduction of airway pressure.

In addition to the NEP test event, the pressure and/or the airflow sensors are used for detecting airflow and pressure in the chamber, allowing the calculation of instantaneous alveolar pressure, instantaneous volume and/or other instantaneous pulmonary properties which are based on flow rate and/or pressure measured before, during, and/or after the NEP test event.

Optionally, the aforementioned NEP test and/or Spirometry test may be used to detect the presence and/or absence of EFL, for example by identifying and measuring a difference between a forced vital capacity (FVC) and a slow vital capacity (SVC). In cases where EFL is present, bronchodilators may be used for improving the accuracy of the lung volume measurement. Optionally, if the breathing device 100 detects EFL during the aforementioned NEP test and/or Spirometry test, the control module 108 may instruct the MMI 106 to present an alert and/or a recommendation for using bronchodilators.

Additionally or alternatively, the PFT is a respiratory muscle endurance test that is performed by the breathing device 100 and one of the instantaneous mechanical pulmonary properties is instantaneous respiratory muscle endurance data during a plurality of instants, optionally continuous, of the PFT. As commonly known, endurance testing is performed to respiratory muscles via challenge breathing having varying device resistances. The ventilatory muscles, like other skeletal muscles, may fatigue and result in respiratory failure. The tested endurance reflects the respiratory muscle ability to resist fatigue. It should be noted that patients with various chronic conditions which affect the respiratory muscles frequently demonstrate more abnormal ventilatory muscle endurance than strength. Thus, skeletal muscles endurance may not be inferred from measurements of strength and ventilatory muscle endurance is measured using a variable resistance, see B. G. Nickerson and T. G. Keens, Measuring ventilatory muscle endurance in humans as sustainable inspiratory pressure, J Appl Physiol 52 (3): 768-772, 1982, which is incorporated herein by reference.

Additionally or alternatively, the PFT is a muscle strength test that is performed by the breathing device 100 and one of the instantaneous mechanical pulmonary properties is instantaneous respiratory muscle strength data during a plurality of instants, optionally continuous, of the PFT. An example of a muscle strength test is an explosive muscle strength test in which the mouth pressure is recorded while performing a inspiratory or expiratory effort against an obstructed airway for at least one second, see, A. Ratnovsky, D. Elad, U. Zaretsky and R. J. Shiner, A technique for global assessment of respiratory muscle performance at different lung volumes, Physiol. Meas. 20 (1999) 37-51, which is incorporated herein by reference.

Artificial increase of airway pressure and an estimated decrease in the difference between the airway pressure and the alveolar pressure is detected for reducing or eliminating “choke points” or EFLs. According to Mead J et al. J. appl. Physio. 22:95-108, 1967, the Equal Pressure Point (EPP) or “choke point” represents the airway site where the pleural pressure equals lateral airway pressure. Just downstream from EPP towards the mouth, dynamic compression limiting flow occurs; In such an embodiment, the shutter 104 and/or the chamber 101 may be adjusted to apply various resistance levels during the PFT, which is optionally defined as a ventilatory muscle endurance test, see Nickerson, B. G., and Thomas G. Keens: Measuring ventilatory muscle endurance in humans as sustainable inspiratory pressure. J. Appl. Physiol.: Respirat. Environ. Exercise Physiol. 52(3): 768-772, 1982, which is incorporated herein by reference. The variation of the resistances may be obtained by changing the shutter's occlusion speed, the shutter's caliber, and/or the placement of one or more diffusive elements in the chamber 101 by changing the resistance at the device, and the difference between airway pressure and alveolar pressure during the PFT may be changed.

The higher the device's resistance, the higher the airway and alveolar pressure required in order to sustain a certain airflow rate. In addition, for a given alveolar pressure, the airway pressure is increased and the flow rate decreases with the incrementing of the device's resistance. Optionally, the variation of the device resistances is obtained by allowing the user to change manually the shutter's caliber, the shutter's occlusion speed and/or pattern and/or the placing of diffusive elements. Optionally, the variation of the resistances is controlled by the MMI 106.

It should be noted that the configurable resistance of the device may be used to allow an artificial increase of airway pressure and a decrease in the differences between airway pressure and the alveolar pressure for reducing and/or eliminating “choke points” or EFLs, training of respiratory muscles, challenge testing, and detecting a peak alveolar testing for muscle strength evaluation.

In parallel to the endurance test, the pressure and/or the airflow sensors are used for detecting airflow and pressure in the chamber, allowing the calculation of instantaneous alveolar pressure, instantaneous volume and/or other instantaneous pulmonary properties which are based on the flow rate and/or pressure which are measured before, during, and/or after the endurance test. Such an embodiment allows the physician to link the various stages in the endurance test, and to the estimated instantaneous pulmonary measurements.

In some embodiments of the present invention, the device is designed for estimating pulmonary measurements, optionally instantaneous, in less-cooperative patients, such as preschool children, geriatric, mentally ill, comatose, mechanically ventilated, unconscious and critically ill patients, and animals. As described above, the pressure and flow rate which are measured by the breathing device 100 are taken during a normal respiration cycle of the user. As such, the user's breathing may be spontaneous and therefore little if any cooperation is required. Optionally, the mouthpiece 103 is connected to a mask that may be easily applied and removed, such as a typical air mask. In such an embodiment, the physician or any other caretaker may easily apply and or remove the mask. As described in as described in International Patent Application No. PCT/IL00081/01031 filed on Jul. 27, 2008, which is incorporated herein by reference, the air is occluded for a number of milliseconds in a manner that does not substantially interrupt normal respiration. As such, the breathing device may be used for estimating pulmonary measurements without substantially affecting the quality of breathing. Optionally, the breathing device may be used for monitoring breathing during sleep. In such an embodiment, the outputs of the control module may be recorded for future analysis, used for alerting the user, a physician thereof and/or a monitoring unit if one of the instantaneous pulmonary measurements increases above and/or decreases below a predefined threshold. In such a manner, breathing patterns, patients, such as obstructive sleep apnea patients, may be recorded for diagnosing apneic events, analyzed for predicting apneic events, and/or alerting before and/or after apneic events.

As the accuracy of PFT measurements are frequently dependent on a high level of patient cooperation, any breathing device which facilitates measuring pulmonary measurements without substantial interference in a short, comprehensive, unchallenging procedure would be a definite advantage. The system may therefore be used to obtain more accurate PFTs in geriatric patients, severe Emphysema patients, wounded patients, patients recovering from surgery or suffering from exhaustion, and/or any other group of patient which may be affected and/or tired by long and/or strenuous testing.

As described above, the control module 108 may be connected to a memory that stores the measured pressure, flow rate and/or the calculations which are based thereon. Optionally, the memory is used for storing the outcomes of some or all of the aforementioned PFTs which may be performed using the breathing device. Optionally, the storage allows detecting and estimating an improvement and/or a decline in the medical condition of the patient. Optionally, the stored data allows generating and outputting a report of the patient's medical condition.

According to some embodiments of the present invention, the breathing device 100 is configured for allowing a home user to measure one or more instantaneous pulmonary measurements, for example as described above, and/or to conduct PFTs as autonomous testing which are held without a guidance of a caretaker or a skilled technician. In such an embodiment, the PFTs may be performed by the user and the outcome of the PFTs may be forwarded to a remote client of the caretaker and/or the physician, for example as described above in relation to the communication unit 110. The user may use the PFT to perform daily and/or hourly pulmonary function tests, reducing the need to visit her physician and/or to stay hospitalized for monitoring. Optionally, the MMI 106 is connected to a memory repository that stores media files with PFT instructions. A respective media file is presented to the user during the performance of the PFT, instructing her to respiration via the chamber 101 of the breathing device 100. Optionally, the instructions are provided by a client terminal which is connected to the breathing device 100 via the communication unit 110, for example as described above. The user performs the test using the presented guidance. The media file may include instruction video clips, textual data, illustrations, audio segments that simulates a breathing frequency, and the like.

Optionally, the MMI 106 is configured to instruct the performance of a game and/or a challenge for training the user's respiration and/or improving the user's cooperation and/or motivation to use the device by reducing intimidating aspects of the testing.

Optionally, the MMI 106 includes a display. In such an embodiment, the instructions may be graphically presented as a set of markers that indicates the performance of the user, for example the flow rate of her respiration and the like. For example, markers and lines may be presented to the user which is asked to actively follow respiratory actions in predefined ranges, such as predefined lung volume or airflow levels.

In such an embodiment, the user may receive automatic indicators or be automatically diagnosed by the device 100. The device may be a stationary device that is placed in a clinic, such as a pulmonary function testing lab. The device may allow the user to be served without waiting for or being attended by a technician or a caretaker.

Optionally, the MMI 106 is configured to instruct the user according to a training pattern or a test which is adapted thereto, for example according to her medical information and/or medical history. As used herein, medical information means age, gender, consumed drugs, weight, blood pressure, pathology and pathophysiology. As used herein, medical history means performance of the user in previous pulmonary tests and/or previously estimated instantaneous pulmonary properties and/or measurements.

Optionally, the MMI 106 is designed to present a feedback to the user performances. Optionally, the user is asked to repeat a test in case the test fails to qualify certain predefined criteria. Optionally, the test is recorded by video and/or audio recording units. The recording allows a physician and/or technician to score the user's performances before, during and/or after a PFT is carried out.

As described above, the device 100 may comprise a communication unit for allowing forwarding data to a remote client and/or database. Optionally, the device is connected, via a local client terminal, for example using a USB and/or a Bluetooth™ connection, to a client terminal that forwards the data to the remote client and/or database, for example via the internet. In such an embodiment the user may use the device at home, at a clinic, outdoors, for example in a vehicle, and/or before, during or after sport activities. In such an embodiment the data may be sent to a central terminal that processes the data and generates alerts and/or reports accordingly. The ability to use the device outdoors and/or from home allows the user to estimate her pulmonary function in different environmental conditions, such as different weather conditions, pollution levels, presence of dust, humidity levels, and/or aerosol conditions. In such a manner the device may be used for estimating instantaneous pulmonary properties and/or measurements in different environmental conditions. Such estimation may allow the physician to monitor and sometimes even diagnose a variety of respiratory disorders particularly those affecting airflow.

Optionally, the breathing device 100 is designed to monitor patients with low and/or non-cooperative behavior pattern, such as intensive care patients and patients under general anesthesia, for example during a surgery. In such an embodiment, the breathing device 200 is connected to a mask, for example as described above, and used for estimating instantaneous alveolar pressure, instantaneous volume and/or other instantaneous pulmonary measurements of the patient during some or all of stages of her continuous breathing.

Optionally, the breathing device 100 is designed to monitor hospitalized and ambulatory patients, such as pre and/or post surgical patients, patients undergoing weaning from ventilation support and intubated patients considered for extubation. In such an embodiment, the instantaneous alveolar pressure, instantaneous volume and/or other instantaneous pulmonary properties of the patient are monitored continuously and/or periodically estimated. The estimation is optionally forwarded to a monitoring unit, for example by using the aforementioned communication unit 110. The monitoring unit optionally analyzes the received parameters and alerts the physician and/or the user if needed. The breathing device 100 may be integrated into a common intensive care unit (ICU) and/or added thereto.

Optionally, the breathing device 100 comprises an inhaler 111 and/or any other dispenser that produces a chemical, optionally as a vapor, for example a bronchodilator such as salbutamol or terbutaline, or a corticosteroid to be inhaled. Optionally, the inhaler 111 is connected to a detachable cartridge 112 that contains the dispensed chemical and may be replaced when needed. Optionally, the inhaler 111 is connected to a direct supply of oxygen or a regulator or oxygen supply. In such an embodiment, the control module 108 may control the amount of the chemical which is dispensed into the chamber 101 according to the alveolar pressure, volume and/or pulmonary property which are measured by the device 100. In such a manner, the portion which is inhaled by the patient is adapted to current pulmonary indications which have been measured using the breathing device 100. Optionally, the device may be used for dispensing bronchodilators during one or more of the PFTs for dilating the bronchi. The bronchodilators may be applied to regulate the respiration of the patient and/or to improve the accuracy of the respective PFT. In use, the shutter 104 and the ventilation valve 301 may be open to allow the user to inhale the dispensed amount of chemical vapor.

Optionally, the breathing device 100 may implement bronchial challenge testing. In such an embodiment, a provoking agent such as an allergen, methacholine, histamine and various agents normally present in the user's environment or work condition is dispensed for provoking a response. Such a bronchial challenge testing may be used for diagnosing patients with Occupational Lung Disease, such as airway hyperresponsiveness, such as found in Asthma, see Anderson S D., Challenge tests to assess airway hyperresponsiveness and efficacy of drugs used in the treatment of asthma, J Aerosol Med. 9(1):95-109. 1996), which is incorporated herein by reference.

As described above, the breathing device 100 may be used to perform one or more tests for assessing instantaneous pulmonary measurements based on instantaneous alveolar pressure and/or volume. In order to provide a more comprehensive assessment of the medical condition of the patient during the tests, the breathing may be connected, integrally or electronically, to additional biological measurement units that optionally provide instantaneous indications about respective biological parameters of the user. Optionally, the assessment of the instantaneous pulmonary measurements is adjusted according to the respective biological parameters.

Optionally, the additional biological measurement units include one or more of the following:

1. A pulsometer for measuring the heart rate of the user;

2. A Pulse Oximeter;

3. A sphygmomanometer for measuring the blood pressure of the user;

4. Image and/or audio sensors for recording the assessment. The image and/or audio sensors may be used for recording the breathing of the user. Optionally, the recording may be used for assuring the quality of a test. Optionally, the recording is forwarded to a remote client terminal, for example using the communication unit, to allow a physician to monitor the test and/or the user performances

5. A thermometer for measuring the body temperature of the patient;

6. A perspiration sensor, such as a humidity sensor for measuring perspiration; and

7. A system for estimating and/or monitoring brain and/or nerve activity, such as electroencephalography (EEG) or electromyography (EMG) and functional magnetic resonance imaging (fMRI).

According to some embodiments of the present invention, the breathing device 100 is used for assessing and/or monitoring pulmonary manifestations of cardiac disorders, such as coronary artery disease, cardiomyopathy, valvular disorders, such as mitral valve disease, and congestive heart failure (CHF). In such an embodiment, the assessed pulmonary measurements, which may be instantaneous, such as airway resistance, respiratory muscle strength and endurance, expiratory flow limitation detection, pulmonary volume, density related pulmonary volume changes, spirometry and respiratory system compliance are used for the assessment, see Brian K. Gehlbach, MD and Eugene Geppert, MD, The Pulmonary Manifestations of Left Heart failure, Chest 125; 669-682. 2004, P. Agostoni et al., Gas diffusion and alveolar-capillary unit in chronic heart failure, Eur Heart J 27:2538-2543. 2006, Szollosi, B. R. Thompson, H. Krum, D. M. Kaye, and M. T. Naughton Impaired Pulmonary Diffusing Capacity and Hypoxia in Heart Failure Correlates With Central Sleep Apnea Severity Chest 134:67-72. 2008 which are incorporated herein by reference

It is expected that during the life of a patent maturing from this application many relevant apparatuses and methods will be developed and the scope of the term a shutter, pressure sensor, an airflow sensor, and a control module is intended to include all such new technologies a priori. As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for determining at least one instantaneous pulmonary measurement, comprising:
a) providing a plurality of instantaneous mechanical pulmonary properties of a user in at least one identified respiratory instant during at least one respiration cycle;
b) correlating between said plurality of instantaneous mechanical pulmonary properties; and
c) determining at least one instantaneous pulmonary measurement in said at least one identified respiratory instant according to said correlation during said at least one respiration cycle.

2. The method of claim 1, wherein a first of said instantaneous mechanical pulmonary properties is one of an instantaneous alveolar pressure or an instantaneous pulmonary volume.

3. (canceled)

4. The method of claim 1, wherein said a)-c) are performed without cooperation of said user.

5. The method of claim 1, wherein said at least one respiration cycle are tidal breathing cycles.

6. The method of claim 1, further comprising outputting said at least one instantaneous pulmonary measurement during said at least one respiration cycle.

7. The method of claim 1, wherein said at least one instantaneous pulmonary measurement comprises an estimation of respiratory system compliance in said at least one identified respiratory instant.

8. The method of claim 7, wherein said at least one respiration cycle comprising an exhalation stage and an inhalation stage, said determining comprises determining at an expiratory respiratory system compliance estimation based on said exhalation stage and an inspiratory respiratory system compliance estimation based on said inhalation stage and calculating a ratio between said expiratory and inspiratory respiratory system compliance estimations.

9. The method of claim 7, wherein said at least one respiration cycle is a tidal breathing respiration cycle.

10. The method of claim 7, wherein said user is in a static respiratory state during said at least one respiration cycle.

11. The method of claim 1, wherein said at least one instantaneous pulmonary measurement comprises an estimation of a pulmonary airway resistance in said at least one identified respiratory instant.

12. The method of claim 1, wherein at least one of said plurality of instantaneous mechanical pulmonary properties is: a continuous testing data of a pulmonary function test (PFT) in respective said at least one identified respiratory instant, or a negative expiratory pressure (NEP) test result.

13. (canceled)

14. The method of claim 12, wherein said PFT is a spirometry test, wherein a first of said instantaneous mechanical pulmonary properties comprising at least one of a volume-time curve and a flow-volume loop

15-19. (canceled)

20. The method of claim 1, wherein said a) comprises allowing a user to breathe during said at least one respiration cycle via a conduit and measuring a flow rate in of conduit for estimating said instantaneous pulmonary volume.

21. The method of claim 20, wherein said user performs said at least one respiration cycle according to an endurance test, a second of said plurality of instantaneous mechanical pulmonary properties is an endurance test value and said a) comprising adjusting a resistance element in said conduit for applying various resistance levels on an airflow induced by said breathing.

22-24. (canceled)

25. The method of claim 1, further comprising assessing a pulmonary manifestation of at least one of a cardiac disorder, respiratory muscle strength disorder, and a pulmonary disorder according to said instantaneous pulmonary measurement.

26-36. (canceled)

37. An apparatus for determining at least one instantaneous pulmonary measurement, comprising:
a housing defining a chamber having at least one of a mouthpiece and a mask configured for allowing a user to exhale and inhale in at least one respiration cycle via said chamber;
at least one sensor positioned in said chamber and configured for measuring a plurality of mechanical pulmonary properties according to said exhaling and inhaling;
a control module configured for generating an instantaneous pulmonary volume and an instantaneous alveolar pressure in a plurality of respiratory instants of said at least one respiration cycle and calculating a respiratory system compliance estimation in each said respiratory instant accordingly; and
an output unit configured for outputting said respiratory system compliance during said at least one respiration cycle.

38. The apparatus of claim 37, wherein said housing is a handheld housing.

39. The apparatus of claim 37, wherein said control module configured for calculating an airway resistance in each said respiratory instant according to said instantaneous pulmonary volume and said instantaneous alveolar pressure, output unit configured for outputting said airway resistance.

40. The apparatus of claim 37, further comprising a communication unit configured for establishing a communication session with a terminal and forwarding at least one of said respiratory system compliance, said pulmonary volume, and said instantaneous alveolar pressure to a client terminal thereto.

41. (canceled)

42. The apparatus of claim 37, wherein said apparatus is adapted for veterinary use.
